(19) United States
(12) Patent Application Publication
(10) Pub. No.: US 2023/0190666 A1
(43) Pub. Date: Jun. 22, 2023

(54) A PARTICLE ENCAPSULATING HYDROPHILIC OR AMPHIPHILIC BIOLOGICAL COMPOUNDS

(71) Applicants: SPHERA ENCAPSULATION S.R.L, Dossobuono (IT); HUDSON RIVER BIOTECHNOLOGY B.V., Wageningen (NL)

(72) Inventors: Anthony Rosa, Povegliano Veronese (IT); Gianni Zoccatelli, Bussolengo (IT); Alberto Natali, Amsterdam (NL); Francesca Zanoni, Cerro Veronese (IT); Giulia Dona, Ronco all'Adige (IT); Ferdinance C.O. Los, Bussum (NL); Martina Vakarelova, Verona (IT)

(21) Appl. No.: 17/926,565

(22) PCT Filed: May 19, 2021
(86) PCT No.: PCT/EP2021/063348

§ 371 Date: Nov. 18, 2022

(30) Foreign Application Priority Data
May 20, 2020 (IT) 102020000011803

(51) Int. Cl. A61K 9/51; A61K 9/19; A61K 8/02; A61Q 19/00

(52) U.S. Cl. CPC A61K 9/5192; A61K 9/5169; A61K 9/5161; A61K 9/19; A61K 8/0245; A61Q 19/00; A61K 2800/413; A61K 2800/412; A61K 2800/654; A61K 2800/624

(57) ABSTRACT

Method for encapsulating a hydrophilic or amphiphilic biological compound, particles obtained by said method, compositions comprising them and uses thereof are disclosed.

### FIELD OF THE INVENTION

The present invention is in the field of particles encapsulating hydrophilic or amphiphilic biological molecules, compositions comprising same, processes of preparing such particles and compositions and uses thereof.

Specifically, the invention concerns the encapsulation of hydrophilic or amphiphilic biological, including biologically active, compounds that enables the delivery of these compounds inside living cells, including cells enclosed by a cell wall, while maintaining the integrity and activity of these compounds.

### BACKGROUND OF THE INVENTION

The efficacy of many bioactive agents is based on their ability to reach the selected target sites and remain present in effective concentrations for sufficient periods of time to accomplish the desired biological activity. In order to do so, the biological compounds, including biological compounds, or bioactive agents, must be delivered inside different types of cells, from mammalian, yeast, bacteria to plant cells and whole organisms. In particular, living cells, specifically cell types containing cell walls, such as plant cells, are notably recalcitrant when it comes to the intracellular delivery of molecules. Nanoparticle/nanoencapsulation formulations have been described as efficient methods of intracellular delivery, but to our knowledge not for delivery through cell walls of.

At present different methods for intracellular delivery are available, such as chemical transfection (e.g. via PEG—polyethylene glycol), electroporation, biolistic transfer, and nano-encapsulation. Of these, only biolistic is able to pass through a plant cell wall but it is highly inefficient.

One type, among the most used methods for the delivery of such molecules, are viral vectors but this kind of approach requires difficult customization for every target and raises concerns about the risk of immunogenicity and hazardous integration events.

At present, different nanoparticle delivery methods are available, such as incorporation or encapsulation in core shell nanoparticles. Specifically, among the non-viral approaches, conventional encapsulation techniques emerging as promising alternatives, are in fact lipid-based encapsulation techniques, since lipidic particles have not shown many problems in packaging hydrophilic or amphiphilic biological compounds. Nevertheless, said conventional lipid-based techniques, employing lipidic particles, have the main disadvantage that they cannot pass through the plant cell wall.

Furthermore, the lipid-based platforms, polymeric nano-carriers and gold-nanoparticles methods are characterized by the use of very expensive ingredients, low loading capacity, scarce release efficacy, low stability and are very difficult to implement to larger scale protocols.

Moreover, all the above-mentioned methods are not applicable to large molecules, such as enzyme complexes, nucleic acid sequences and large proteins.

In view of all the above, there is an increased need to overcome the limitations of conventional encapsulation techniques, and to find methods of providing solid or liquid, hydrophilic active substances such as proteins or nucleic acid molecules for their controlled release without losing their activity.

As above, there is still a need of providing new methods directed to both hydrophilic and amphiphilic biological compounds, including biologically active compounds, such as DNA, RNA, enzyme and peptides, able to overcome the limitations of conventional encapsulation techniques and to assure the delivery and controlled release of said molecules inside different types of living cells, from plant cells, yeast, bacteria, to animal cells, such as mammalian cells, while keeping them stable and active.

As a consequence, a primary object of the present invention is the delivery of hydrophilic and amphiphilic biological compounds, including biologically active compounds, in different kinds of cells while maintaining the integrity and activity of these biological compounds.

### SUMMARY OF THE INVENTION

The inventors found that through the application of a specific solvent, with a specific dielectric constant, to specific proteins allowed to permanently modify the conformation of said proteins and to form a protein-based shell, also partially comprising the biological hydrophilic or amphiphilic compound, thus obtaining a matrix-type or mold-type particle.

Therefore, the solution proposed herein for the aforementioned object, is a method for encapsulating a hydrophilic or amphiphilic biological compound, comprising the steps of:

a. forming a two-phase solution by solubilizing the hydrophilic or amphiphilic biological compound and a protein in water to form a solution, and mixing the protein solution and a solvent, thereby obtaining a two-phase solution;
b. emulsifying the two-phase solution in order to obtain an emulsion; and
c. evaporating the solvent from the emulsion;
thereby obtaining a particle comprising: (i) a protein-based shell and (ii) a hydrophilic or amphiphilic biological compound,
wherein said hydrophilic or amphiphilic biological compound is at least partially included in said protein-based shell, and
wherein said solvent of step a. has a dielectric constant at 20-25° C. in the range from 1.5 to 15.

In fact, without being bound to any theory, the inventors have surprisingly found that by using a specific solvent, being non-water miscible and having a specific dielectric constant, in combination with a protein, it was possible to obtain a new type of particle, specifically in the form of a matrix-type or mold-type particle, wherein the biological hydrophilic or amphiphilic compound is encapsulated in the protein-based shell or it is at least partially included in the protein-based shell. Said specific solvent, due to its polarity properties, connected to the dielectric constant, was able to act as “nucleation center” for the formation of the particles, and after its evaporation allowed to obtain the particular matrix-type or mold-type particles of the invention, where the biological compound resides both inside and along the boundaries of the particle. The combination of the specific solvent and protein allows for specific molecular interactions, which distributes the biological hydrophilic or amphiphilic compound throughout the structure of the particle, specifically including it in the shell and not only in its center. The final particle thus obtained can pass through the cellular plasma membrane to deliver, and afterwards release, the biological compounds, in several types of living cells, including the most recalcitrant ones such as plant cells containing cell walls.

Said solvent, having a dielectric constant at 20-25° C. in the range from 1.5 to 15 can be conveniently chosen among physiologically accepted solvents, more specifically solvents acceptable for pharma, nutraceutical, and cosmetic applications, such that the matrix-type particle of the invention can be used not only in agrochemical applications, but also in pharmaceutical, nutraceutical and cosmetical fields.

The method for encapsulating a hydrophilic or amphiphilic biological compound allowed to obtain a specific particle, comprising: (i) a protein-based shell and (ii) a hydrophilic or amphiphilic biological compound.

wherein said hydrophilic or amphiphilic biological compound is at least partially included in said protein-based shell and retains its biological activity.

The inventors surprisingly found that, through the method of the present invention, they could obtain a specific particle with a small diameter, which is able to pass through the eukaryotic cell wall, specifically a plant cell wall, and deliver the hydrophilic or amphiphilic biological compound inside living cells, specifically said plant cells, while maintaining the integrity and activity of these biological compounds.

Therefore, in a preferred and advantageous aspect, the invention relates to a particle having a diameter in the range from 1 to 60 nm, preferably from 5 nm to 60 nm, more preferably 5 nm to 50 nm as measured through Dynamic Light Scattering (DLS).

The inventors further found that, through the method of the present invention, they could also tune the particle diameter, thus obtaining a specific particle with a larger diameter, which is able to pass through living cells without any cellular wall, namely eukaryotic cells, specifically animal cells, more specifically mammalian cells, while maintaining the integrity and activity of these biological compounds.

Therefore, in a further preferred and advantageous aspect, the invention relates to a particle having a diameter in the range from 70 to 700 nm, preferably from 100 to 500, more preferably from 100 to 300 nm, still more preferably from 100 to 200 nm as measured through Dynamic Light Scattering (DLS).

According to another aspect, there are provided uses of said particle obtainable by the method of the invention for the delivery of at least a hydrophilic or amphiphilic biological compound inside living cells, including cells enclosed by a cell wall.

According to further aspect, there is provided a composition comprising a plurality of the obtained particles.

In an advantageous aspect, the composition has a polydispersity index of 0.05 to 0.7, preferably in the range from 0.2 to 0.6, preferably in the range from 0.2 to 0.4 as measured through Dynamic Light Scattering (DLS).

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The invention is described in detail below with reference to the Figures, specifically:

FIG. 1 is a scheme representing the method of the invention, which leads to the formation of the particles (i.e BSA-WPH Ps⁻ and e BSA-WPH Ps⁺ with the additional coating of chitosan) of the invention.

FIGS. 2A and 2B are representations of different particles; FIG. 2A represents core-shell particles (c-Ps) structure of the prior art; FIG. 2B represents the matrix type or mold type particles (m-Ps) structure of the invention;

FIGS. 3A and 3B show microscopic images of FIG. 3A the encapsulated BSA in freeze dried BSA-WPH Ps⁻ particle of the invention; and FIG. 3B the encapsulated BSA in freeze dried BSA-WPH Ps+ particle of the invention;

FIG. 4A shows that after treatment with BSA-WPH PS⁺ it was not possible to obtain protoplasts from the preparation, this was possible when cells were treated with BSA-WPH PS⁻, FIG. 4B shows the isolated protoplasts from the treated cells with BSA-WPH Ps⁻ analyzing their cellular delivery;

FIGS. 5A and 5B provide fluorescence and transmitted light microscope images, demonstrating that delivery of BSA in the BSA-WPH Ps particles of the invention depends on the charge of the Ps; FIG. 5A BSA-WPH Ps⁺ does not enable intracellular delivery; FIG. 5B BSA-WPH Ps⁻ does enable intracellular delivery;

FIG. 6 provides fluorescence and transmitted light microscope images of the delivery of fluorescently labeled BSA-WPH Ps⁻, for cells from Nicotiana tabacum in cell suspension;

FIGS. 7A, 7B and 7C show characteristics of the different particles produced according to Example 1, and having a shell comprising GFP (without or with a nuclear localization signal [NLS]) as the hydrophilic biological compound and different proteins as protein-based shell, such as WPH (OPTIPEP commercial name), fava protein isolate, potato isolate protein, and soya protein isolate; graphs reporting FIG. 7A the z-average, FIG. 7B number (where the distribution by number shows the relative proportion of differently sized particles considering their number in solution) and FIG. 7C the PDI index.

FIGS. 8A and 8B provide fluorescence and transmitted light microscope images of the nanoparticles and the cells of Nicotiana tabacum cells having intact plant cell walls; specifically FIG. 8A provides an image of the basal fluorescence of the nanoparticles, thus the control, intended as non-treated cells, to check the background fluorescence, and FIG. 8B provide an optical microscope image of the empty nanoparticles (NPs) with WPH in the shell, without GFP, in suspension with Nicotiana tabacum cells having intact plant cell walls;

FIG. 9 provides fluorescence and transmitted light microscope images of the bioavailability of the non-encapsulated GFP (naked GFP) in suspension with Nicotiana tabacum cells having intact plant cell walls;

FIG. 10 provides fluorescence and transmitted light microscope images demonstrating intracellular delivery of GFP protein encapsulated in the GFP-WPH NPs⁻ particles of the invention, in suspension with Nicotiana tabacum cells having intact plant cell walls; this figure indicates that the GFP retains its biological integrity (i.e., GFP fluorescence preservation) throughout the process after being delivered inside a cell through the plant cell wall.

FIG. 11 provides fluorescence and transmitted light microscope images demonstrating intracellular delivery of GFP protein with a nuclear localization signal (NLS) encapsulated in the GFP-WPH NPs⁻ particles of the invention, left over night in suspension with Nicotiana tabacum cells having intact plant cell walls, then washed and observed through an optical microscope equipped with fluorescence filter, and subsequent nuclear localization of the signal resulting from bioactivity, i.e., interaction of the NLS with the host cell's natural intracellular trafficking machinery.

FIG. 12 provides fluorescence and transmitted light microscope images of the intracellular delivery of GFP encapsulated in the particle Fava-GFP NPs⁻ of the invention, in suspension with Nicotiana tabacum cells having intact plant cell walls;

FIGS. 13A and B provides fluorescence and transmitted light microscope images of the intracellular delivery of GFP encapsulated in the particle Soya-GFP NPs⁻ (GFP-SP NPs⁻) of the invention, specifically in suspension with Nicotiana tabacum cells having intact plant cell walls (FIG. 13A) and in cells calli (FIG. 13B);

FIG. 14A provides optical microscope images of the bioavailability of the naked DNA (naked p-RAP) in suspension with Nicotiana tabacum cells;

FIG. 14B provides optical microscope images of the bioavailability of the DNA encapsulated in the particle DNA-WPH Ps⁻ of the invention (pRAP-NPs (DNA-WPH Ps⁻)), in suspension with Nicotiana tabacum cells;

FIG. 15A provides optical microscope images of the bioavailability of the empty cellulase-GFP Ps nanoparticles;

FIG. 15B provides optical microscope images of the bioavailability of the 0.5 mg/ml cellulase enzyme encapsulated in the particle cellulase-GFP Ps⁻ of the invention, with Nicotiana tabacum in calli;

FIG. 16 represents a proposed scheme of the formation of enzyme (cellulase and amylase)-WPH-Ps of the invention;

FIG. 17 provides a comparison of the enzymatic activity of the cellulase enzyme alone, cellulase enzyme dissolved in whey protein, encapsulated cellulase-WPH Ps⁻ (NPs) of the invention, and the cellulase-NPs broken again after encapsulation, when in contact with sugar. The activity was tested through the fenol sulfuric assay test;

FIG. 18 provides a comparison of the enzymatic activity of the amylase enzyme alone, amylase enzyme dissolved in whey protein, encapsulated amylase-WPH Ps⁻ of the invention, and the amylase-NPs broken again after encapsulation, when in contact with sugar. The activity was tested through the fenol sulfuric assay test;

FIG. 19 provides optical microscope image of the basal fluorescence of the non-treated macrophages J77A.1; and

FIG. 20 provides optical microscope images of the suspension of J77A.1 cells treated with the nanoparticles (NPs) with WPH in the shell and containing GFP (GFP-WPH Ps⁻).

### DETAILED DESCRIPTION OF THE INVENTION

Therefore, the present invention relates to a method for encapsulating a hydrophilic or an amphiphilic biological compound, comprising the steps of:

a. forming a two-phase solution by solubilizing the hydrophilic or an amphiphilic biological compound and a protein in water to form a protein solution, and mixing the protein solution and a solvent, thereby obtaining a two-phase solution;
b. emulsifying the two-phase solution in order to obtain an emulsion;
c. evaporating the solvent from the emulsion;
thereby obtaining a particle comprising (i) a protein-based shell (ii) a hydrophilic or an amphiphilic biological compound,
wherein said hydrophilic or amphiphilic biological compound is at least partially included in said protein-based shell,
wherein said solvent of step a., has a dielectric constant at 20-25° C. in the range from 1.5 to 15.
In the present invention, when using the following terms:
“biological compound” or “hydrophilic or amphiphilic biological compound” refers to a biological macromolecule, including proteins and nucleic acids and combinations thereof. Said biological compound can be a biologically active compound or a non-biologically active compound. Said biologically active compound refers to a compound that exerts a biological or chemical change in an organism or inside a living organism or cell thereof, including but not limited to mammalians, vascular plants, non-vascular plants (eukaryotic algae, mosses), fungi, yeast and prokaryotic organisms (bacteria, cyanobacteria, etc.). In some embodiments, the biological hydrophilic or amphiphilic compound has a MW smaller than 1,000 Da;
“hydrophilic compound or hydrophilic biological compound” it is intended a biological compound which, when introduced into water at a concentration of at least 1%, at least 5%, at least 10%, by weight, results in a macroscopically homogeneous solution;
“core-shell type particle” refers to a particle wherein the biological compound is encapsulated only in the core, without being included in the shell, nor even in the surface of the particle;
“matrix-type particle” or “mold-type particle” refers to a particle wherein the active principle is distributed not only in the core, but also in the structure of the particle, specifically the protein-based shell, with the possibility that some biological compound is exposed to the surface;
“protein hydrolysate” it is intended all hydrolyzed products of proteins prepared by using a proteolytic enzyme preparation, a microorganism containing suitable proteolytic activity or acid hydrolysis or any combination thereof and having serum lipid profile improving effect. Commercially available hydrolysates can be used, or hydrolysates can be prepared. In some embodiments, hydrolysates have a molecular weight of 300-100000 Da, 500-50000 Da;
“whey protein” it is intended, in some embodiments, a product comprising at least 80%, 85%, 90% of whey proteins;
“living cells” it is intended a cell which is able to: —respond to changes in its environment, —grow and develop across its lifespan, —reproduce, or make copies of itself, —have metabolism, —maintain homeostasis, or keep its internal environment the same regardless of outside changes, —pass on traits to its offspring.

Specifically, living cells can be selected from “cells without cell walls” (such as animal cells, certain micro-organisms, and protoplasts) and “cells with cell walls” (typical plant cells, certain micro-organisms), more specifically from procaryotes, such as bacteria and archaea, more specifically such as bacteria cells and yeast cells, to eucaryotes such as eukarya, including plant cells, fungi, and animal cells, specifically mammalian cells.
“zeta potential” it is intended to a scientific term for electrokinetic potential in colloidal systems. In the colloidal chemistry literature, it is usually denoted using the Greek letter zeta, hence ζ-potential. Zeta potential is a measure of the magnitude of the repulsion or attraction between particles. Zeta potential is an index of the magnitude of interaction between colloidal particles and measurements of zeta potential are used to access the stability of colloidal systems. In aqueous media, the pH of the sample affects its zeta potential. For example, if alkali is added to a suspension with a negative zeta potential the particles tend to acquire more negative charge. If sufficient acid is added to the suspension, then a point will be reached where the charge will be neutralized. Further addition of acid will cause a buildup of positive charge.
“about” refers to ±10%.
“comprises”, “comprising”, “includes”, “including”, “having” and their conjugates it is intended “including but not limited to”.
“consisting of” it is intended “including and limited to”.
“exemplary” it is intended “serving as an example, instance or illustration”. Any embodiment described as “exemplary” is not necessarily to be construed as preferred or advantageous over other embodiments and/or to exclude the incorporation of features from other embodiments.
“optionally” it is intended “is provided in some embodiments and not provided in other embodiments”. Any particular embodiment of the invention may include a plurality of “optional” features unless such features conflict.
the singular form “a”, “an” and “the” include plural references unless the context clearly dictates otherwise. For example, the term “a compound” or “at least one compound” may include a plurality of compounds, including mixtures thereof.
“method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.
“treating” includes abrogating, substantially inhibiting, slowing or reversing the progression of a condition, substantially ameliorating clinical or aesthetical symptoms of a condition or substantially preventing the appearance of clinical or aesthetical symptoms of a condition.
“cationic polymer” refers to naturally and synthetically derived cationic polymers.
“chitosan derivative” is used wherein one or more hydroxyl groups and/or one or more amine groups have been modified (e.g., acetylated, alkylated or sulfonated chitosans, thiolated derivatives).

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

According to a preferred and advantageous aspect, the encapsulation method of the invention is non-destructive and reversible, at least for the industrial enzymes.

Advantageously, the encapsulation method of the invention is performed, using chemicals that are acceptable to pharma, food, cosmetics, etc

The method comprises a step a. of mixing the protein solution and a solvent.

Said solvent of step a. has a dielectric constant at 20-25° C. in the range from 1.5 to 15, preferably from 1.8 to 6.02.

Preferably, said solvent of step a., having a dielectric constant at 20-25° C. in the range from 1.5 to 15, is selected from the group consisting of ethyl acetate (6.02), dichloromethane (8.93), pentane (1.84), chloroform (4.81), 1,4 dioxane (2.25), benzene (2.27), toluene (2.38), N-pentane (1.84), N-hexane (1.88), cyclohexane (2.02).

More preferably, said solvent of step a., has a dielectric constant at 20-25° C. in the range from 1.8 to 6.02.

Still more preferably, said solvent of step a., is ethyl acetate, having a dielectric constant of 6.02.

All the dielectric constant described were as stated by the supplier and eventually checked via known techniques, such as dielectric probe or oscilloscope or microwave dielectrometer.

Preferably, said solvent of step a., having a dielectric constant at 20-25° C. in the range from 1.5 to 15, is in a weight ratio between protein:solvent in the range 10:0.5 to 8:1.5.

More preferably, said solvent of step a. is in a weight ratio between protein:solvent of 9:1.

In an advantageous and preferred embodiment, the method comprises a further step e. of adding a cationic polymer, thus coating the particle, thus encapsulating the particle.

Preferably, said cationic polymer is a cationic polysaccharide.

Non-limiting examples of cationic polysaccharide polymers include: cationic celluloses and hydroxy ethyl celluloses; cationic starches and hydroxyalkyl starches; cationic polymers based on arabinose monomers such as those which could be derived from arabinose vegetable gums; cationic polymers derived from xylose polymers found in materials such as wood, straw, cottonseed hulls, and corn cobs; cationic polymers derived from fucose polymers found as a component of cell walls in seaweed; cationic polymers derived from fructose polymers such as inulin found in certain plants; cationic polymers based on acid-containing sugars such as galacturonic acid and glucuronic acid; cationic polymers based on amine sugars such as galactosamine and glucosamine; cationic polymers based on 5 and 6 membered ring polyalcohols; cationic polymers based on galactose monomers which occur in plant gums and mucilages; cationic polymers based on mannose monomers such as those found in plants, yeasts, and red algae; cationic polymers based on the galactomannan copolymer known as guar gum obtained from the endosperm of the guar bean.

More preferably, said cationic polymer is a cationic polysaccharide, still more preferably said cationic polysaccharide is chitosan or a derivative thereof.

Advantageously, the chitosan or a derivative thereof is characterized by having a low molecular weight, such as of less than 90 kDa, less than 75 kDa, less than 50 kDa, less than 30 kDa, or less than 15 kDa.

Still advantageously, a derivative of chitosan is used wherein one or more hydroxyl groups and/or one or more amine groups have been modified (e.g., acetylated, alkylated or sulfonated chitosans, thiolated derivatives), with the aim of increasing the solubility of the chitosan or increasing the adhesive nature thereof.

In an advantageous and preferred embodiment, the method comprises the step e. of adding a cationic polymer, thus coating the particle, thus encapsulating the particle, prior to step d. of drying the evaporated emulsion.

In another advantageous and preferred embodiment, the method comprises the step e. of adding a cationic polymer, thus coating the particle, thus encapsulating the particle, after step d. of drying the evaporated emulsion.

According to the method of the invention, it is present a step a. of forming a two-phase solution by solubilizing the hydrophilic or an amphiphilic biological compound and a protein in water to form a protein solution.

Preferably, the hydrophilic or amphiphilic biological compound of step a. is a biological compound selected from the group consisting of an oligonucleotide, a nucleic acid, a protein, peptides, hormones, an enzyme or any combination thereof.

More preferably, the hydrophilic or amphiphilic biological compound is selected from BSA (bovine serum albumin), GFP (green fluorescent protein), DNA, an antisense RNA, messenger RNA, CRISPR enzyme, CRISPR enzyme-guide RNA complex, cellulase enzyme, amylase enzyme.

In an advantageous embodiment, the hydrophilic or amphiphilic biological compound of step a. is in a suspension. Preferably, the hydrophilic or amphiphilic biological compound of step a. is a suspension in a solvent.

Advantageously, the protein of step a. may be an extract selected from an animal protein, a plant protein, or an algae protein. The protein extract may be selected from: a purified protein, a concentrated protein, an isolated protein fraction, a protein hydrolysate, or any combination thereof.

Preferably, the protein of step a. is selected from whey protein, soya protein, pea protein, fava bean protein, and potato protein or any combination thereof.

More preferably, the protein of step a. is in the form of a protein hydrolysate.

In fact, the inventors noticed that hydrolyzed proteins could be used in the method of the invention to obtain said specific particle, due to their low molecular weight, which allows them to achieve smaller particle sizes, in the range of nanoparticles. According to this preferred embodiment, advantageously the particle of the invention is more prone to penetration in the case of plant cells membranes.

Still more preferably, the protein of step a. is whey protein, fava bean protein and soya protein, still more preferably is whey protein hydrolysate.

Preferably, said step a. is carried out by using an ultra-sonicator for a time duration in a range from 5 seconds to 30 minutes, preferably 1 minute to 15 minutes, more preferably 1 minute to 10 minutes.

More preferably, said step a. of mixing a solvent with a protein by using an ultra-sonicator is carried out for a time duration selected from the group consisting of 1, 3, 2 and 5 minutes.

More preferably, said step a. of mixing a solvent with a protein by using an ultra-sonicator is carried out for a time duration of 5 minutes.

Still more preferably, said step a. of mixing a solvent with a protein is carried out by sonicating the solution for a time duration of 5 minutes at a potency of 10 W, through a Microson ultrasonic cell disruptor XL.

Advantageously, said step a. of mixing is carried out under 5° C. (e.g., on ice) so to avoid the overheating of the solution.

In an advantageous embodiment, said step a. of mixing a solvent with a protein is carried out by using a high shear homogenizer.

Advantageously, said step a. of mixing is carried out by using a combination of high shear homogenizer and an ultra-sonicator.

The method according to the present invention, comprises a step b. of emulsifying the two-phase solution, obtained in step a. in order to obtain an emulsion.

Preferably, said step b. of emulsifying is carried out by using an ultra-sonicator for a time duration in a range from 5 seconds to 30 minutes, preferably 1 minute to 15 minutes, more preferably 1 minute to 10 minutes, including any range therebetween.

More preferably, said step b. of emulsifying is carried out or a time duration selected from the group consisting of 1, 3, 2 and 5 minutes.

More preferably, said step b. of emulsifying using an ultra-sonicator is carried out for a time duration of 5 minutes.

Still more preferably, said step b. emulsifying is carried out by sonicating the solution for a time duration of 5 minutes at a potency of 10 W, through a Microson ultrasonic cell disruptor XL.

Advantageously, said step b. of emulsifying is carried out under 5° C. (e.g., on ice) so to avoid the overheating of the solution.

In some an advantageous embodiment, said step b. of emulsifying is carried out by using a high shear homogenizer.

Advantageously, said step b. of emulsifying is carried out by using a combination of high shear homogenizer and an ultra-sonicator.

These conditions enable the formation of finer particles. In fact, the inventors deem that by tuning the potency and the time of sonication of step b., it is possible to obtain different ranges of particles diameter, as will be shown below.

According to the method of the invention, it is needed a step c. of evaporating the solvent from the emulsion, in order to form and stabilize the NPs structure.

In an advantageous embodiment, the step c. of evaporating a solvent is done by using a nitrogen flow, nitrogen flow in the dark, an evaporator, a rotary evaporator such as circulation evaporator, falling film evaporator, rising film evaporator, climbing and falling film plate evaporator, multiple-effect evaporator, agitated thin film evaporator air current, or any combination thereof.

Preferably, the step c. of evaporating the solvent is done by nitrogen flow in the dark.

According to a preferred and advantageous embodiment, the method of the invention further comprises a step d. of drying the evaporated particle of step c., comprising (i) a protein-based shell (ii) a hydrophilic or an amphiphilic biological compound, wherein said hydrophilic or amphiphilic biological compound is at least partially included in said protein-based shell, of step c. or the particle coated in step e., comprising (i) a protein-based shell (ii) a hydrophilic or an amphiphilic biological compound and (iii) polysaccharide coating encapsulating the particle, wherein said hydrophilic or amphiphilic biological compound is at least partially included in said protein-based shell.

Advantageously, the drying step d. is selected from the group consisting of spray drying, granulating, agglomerating, freeze drying or any combination thereof, the particles.

Preferably, said step d. of drying is performed by freeze drying the particle obtained in step c.

Freeze drying was chosen in order to stabilized BSA NPs preparations. This method was selected among others (e.g. spray dry and fluid bed) since it is the mildest technique, indicated for the processing of valuable molecules, highly instable to harsh conditions or transportation.

In a particular embodiment, the drying step d. is performed after including low molecular weight molecules, (e.g., maltodextrin Dextrose Equivalent (DE) 19 and glycerol) to the solution, for avoiding high pressure damage to the structure of the particle.

Without being bound to any theory the inventors have surprisingly noticed that the addition of low molecular weight molecules, specifically maltodextrins, to the solution, before proceeding into the drying step, could keep the particles stable. In fact, maltodextrins are deposited around the particles obtained after step c. of the method of the invention, and they act as a filler, thus preventing the pressure generated during the freezing and then freeze-drying process from damaging the particle structure.

In an advantageous and preferred embodiment, the method has an encapsulation yield of 65% to 95%, 60% to 90%, 70% to 90%, 70% to 85%, 75% to 80%, or 75% to 90%, including any range therebetween. In some embodiments, the method has an encapsulation efficacy of 80% to 100%.

In a specific embodiment, the method of the invention allows to obtain a particle having a diameter in the range from 1 to 60 nm, from 5 nm to 60 nm, more preferably 5 nm to 50 nm, including any range therebetween.

In another specific embodiment, the method of the invention allows to obtain a particle having a diameter in the range from 70 to 700 nm, preferably from 100 to 500, more preferably from 100 to 300 nm, still more preferably from 100 to 200 nm, including any range therebetween.

The possibility of obtaining different particles diameters depends on the sonication parameters, employed in step b. of emulsifying, specifically if a sonication of the solution for at least 5 minutes is used, this enables the formation of finer particles.

While by tuning the time of sonication, it is also possible to obtain larger particles diameters, such as the ranges herein described.

According to a second aspect, the present invention provides a particle obtainable by the method above described, comprising:

(i) a protein-based shell; and

(ii) a hydrophilic or an amphiphilic biological compound; and

wherein said hydrophilic or amphiphilic biological compound is at least partially included in said protein-based shell.

In an advantageous embodiment, the particle obtainable by the method of the invention has a diameter in the range from 1 to 60 nm, preferably from 5 nm to 60 nm, more preferably 5 nm to 50 nm, including any range therebetween.

The specific structure of the particle comprising (i) a protein-based shell and (ii) a hydrophilic or an amphiphilic biological compound, wherein said hydrophilic or amphiphilic biological compound is at least partially included in said protein-based shell, in combination with diameters lower than 60 nm, enables said particle to pass through the cell's plasma membrane, deliver, and afterwards release the biological compounds, in several types of cells, specifically living cells, including the most recalcitrant ones, such as plant cells enclosed by a cell wall.

Advantageously, said plant cells are in the form of a cell suspension or calli.

On the other hand, while taking into consideration mammalian cells, specifically macrophages of mice, it is possible to use larger particles, since these types of cells, in the absence of plant cell membranes and plant cell walls, are less reluctant.

Thus, for these type of mammalian cells, only the specific structure of the particle of the invention, comprising (i) a protein-based shell and (ii) a hydrophilic or an amphiphilic biological compound, wherein said hydrophilic or amphiphilic biological compound is at least partially included in said protein-based shell is a key feature, allowing the delivery of said hydrophilic and amphiphilic biological compounds, while entering the mammalian cells, and at the same time to protect the molecules that they are carrying to the site of action.

Thus, in another advantageous embodiment, the particle obtainable by the method of the invention has a diameter in the range from 70 to 700 nm, preferably from 100 to 500, more preferably from 100 to 300 nm, still more preferably from 100 to 200 nm, including any range therebetween.

According to the invention, the particle obtainable by the method of the invention comprises (i) a protein-based shell including at least partially the hydrophilic or amphiphilic biological compound is included in said protein-based shell.

The protein included in the protein-based shell may be an extract selected from an animal protein, a plant protein, or an algae protein. The protein extract may be selected from: a purified protein, a concentrated protein, an isolated protein fraction, a protein hydrolysate, or any combination thereof.

Preferably, the protein-based shell comprises a protein selected from whey protein, soya protein, pea protein, fava bean protein, and potato protein or any combination thereof.

More preferably, said protein is in the form of a protein hydrolysate.

In fact, the inventors noticed that hydrolyzed proteins could be used in the method of the invention to obtain said specific particle, due to their low molecular weight, which allows them to achieve smaller particle sizes, in the range of nanoparticles. Said particle resulted to be surprisingly more prone to penetration, specifically in the case of plant cells membranes.

Even more preferably the protein-based shell comprises a protein selected from whey protein hydrolysate and fava isolate protein, still more preferably the protein-based shell comprises hydrolysate whey protein.

Plant, animal or microbial proteins and/or their mixtures can be used as protein sources for the hydrolysates. Suitable vegetable protein sources are for example soybean protein, wheat protein, wheat gluten, corn protein, oat protein, rye protein, rice protein, rapeseed or canola protein, barley protein, flaxseed protein, potato protein, pea protein, lupin protein, sunflower protein, hemp protein, fava bean protein and buckwheat protein.

In some embodiments, the protein is of animal origin. Suitable animal protein sources are for example milk proteins, such as caseins and whey protein, and their fractions, egg proteins, collagens and gelatins.

Preferably, proteins can be used in different commercially available purified or non-purified forms as source for the hydrolysates.

Advantageously, materials containing these proteins and other major constituents, such as carbohydrates, are used as source for the hydrolysates.

In an advantageous embodiment, the protein extract is a plant protein. In some embodiments, the plant protein is extracted from potato, pea, soy, chickpea, quinoa, wheat, lentils, fava or bean.

In a further preferred embodiment, a protein extract is an animal protein (e.g., a mammal, a bird or an insect).

Preferably, the protein is whey protein.

In a preferred embodiment, the protein-based shell including at least partially the amphiphilic or hydrophilic biological compound is at least partially surrounding said biological compound of at least 25%, preferably at least 35%, more preferably at least 50%, or still more preferably at least 75%, of the total surface of compound.

Preferably, the protein-based shell is in a form of a matrix structure, thus obtaining a matrix-type particle or a mold-type particle, comprising (i) a protein-based shell and (ii) a hydrophilic or an amphiphilic biological compound, wherein said hydrophilic or amphiphilic biological compound is at least partially included in said protein-based shell.

In an advantageous embodiment, the particle comprises 0.1% to 99% (w/w), 0.1% to 90% (w/w), 0.1% to 50% (w/w), 0.1% to 30% (w/w), 0.5% to 30% (w/w) of the protein-based shell, including any range therebetween.

In another advantageous embodiment, the content of the protein-based shell depends on the final concentration of the encapsulated hydrophilic or an amphiphilic biological compound needed. If a low concentration of hydrophilic or an amphiphilic biological compound is needed, the protein-based shell content can be increased up to 99.9%.

According to the invention, the particle obtainable by the method of the invention further comprises (ii) a hydrophilic or amphiphilic biological compound, wherein said hydrophilic or amphiphilic biological compound is at least partially included in said protein-based shell.

Preferably, the hydrophilic or amphiphilic biological compound is a hydrophilic compound selected from the group consisting of an oligonucleotide, a nucleic acid, a protein, such as an enzyme, a peptide, a hormone or any combination thereof.

Preferably, the hydrophilic or amphiphilic biological compound is selected from BSA (bovine serum albumin), GFP (green fluorescent protein), DNA, an antisense RNA, messenger RNA, CRISPR enzyme, CRISPR enzyme-guide RNA complex, cellulase enzyme, amylase enzyme.

Said biological hydrophilic or amphiphilic compound may be effective in various fields including but not limited to pharmaceutical use, cosmetic use, biotechnology use, agricultural use (e.g., herbicides, pesticides, fertilizer), diagnostic and/or theragnostic use.

In a preferred embodiment, the biological hydrophilic or amphiphilic compound is a genome modifying molecule such as a gene silencing molecule or a gene replacement and/or gene insertion molecule or a molecule that makes targeted modifications of the genome, or a messenger RNA encoding such molecule. The genome modifying molecule may be a CRISPR ribonucleoprotein (RNP), zinc finger nuclease (ZFN), transcription activator-like effector nuclease (TALEN), and oligonucleotides (such as small interfering RNA (siRNA), and short hairpin RNA (shRNA)). Each possibility represents a separate embodiment. In some embodiments, the biological compound is at least one CRISPR element, e.g., an RNPs and/or single-guide RNA (gRNA), or DNA encoding such. The RNP may comprise an RNA-guided nuclease and a single-guide RNA (sgRNA). Non-limiting examples of RNA-guided nuclease include: Cas9 (SpCas9, StCas9, SaCas9, ScCas9, and dead Cas9), CasX, CasY, Cas-Phi, Cas12a (Cpf1), Cas13, Cas14 and MAD7.

In a preferred embodiment, the biological hydrophilic or amphiphilic compound is an enzyme. Said enzyme can be selected from the group consisting of carbohydrases (including cellulases, amylases, pectinases and lactases), proteases, lipases, phytases, laccases, polymerases and nucleases.

In an advantageous and preferred embodiment, the hydrophilic or amphiphilic biological compound is provided as a hydrolysate. As depicted herein below, hydrolyzed proteins may be utilized due to their low molecular weight, which allows to achieve smaller particle sizes.

In an advantageous embodiment, the hydrophilic or amphiphilic biological compound is in a suspension. Preferably, the hydrophilic or amphiphilic biological compound is a suspension in a solvent.

Advantageously, the particle comprises 1% to 80% (w/w), 1% to 70% (w/w), 1% to 60% (w/w), 1% to 50% (w/w), 1% to 40% (w/w), 2% to 40% (w/w), 5% to 40% (w/w), 10% to 70% (w/w), 10% to 40% (w/w), 15% to 40% (w/w), 25% to 40% (w/w), 1% to 35% (w/w), 1% to 25% (w/w), 1% to 20% (w/w), 1% to 15% (w/w), 1% to 10% (w/w), 5% to 70% (w/w), 5% to 55% (w/w), 5% to 35% (w/w), 5% to 25% (w/w), 5% to 20% (w/w), 5% to 15% (w/w), or 5% to 10% (w/w), of the hydrophilic or an amphiphilic biological compound.

In an advantageous embodiment of the invention, the particle comprises from 0.1% to 20% of the hydrophilic or an amphiphilic biological compound.

In a further preferred embodiment, the concentration of the hydrophilic or an amphiphilic biological compound in a particle is about 0.01 mg/g to 500 mg/g.

Preferably, the concentration of the hydrophilic or an amphiphilic biological compound in a particle is 0.01 mg/g to 250 mg/g, about 1 mg/g to 100 mg/g, about 1 mg/g to 50 mg/g, about 1 mg/g to 30 mg/g, or about 1 mg/g to 5 mg/g, including any range therebetween, with respect to 1 g of protein-based shell.

In an advantageous and preferred embodiment, the concentration of the hydrophilic or an amphiphilic biological compound in a particle is from 0.01 mg/g to 300 mg/g with respect to 1 g of protein-based shell, preferably from 4 mg/g to 220 mg/g with respect to 1 g of protein-based shell.

According to the invention, the particle obtainable by the method of the invention can further comprise (iii) a cationic polymer, thus forming a coating, encapsulating the particle.

Said cationic polymer is able to interact (e.g., via electrostatic interactions) with at least a portion of a protein-based shell, thus forming a coating.

Preferably, said cationic polymer is a cationic polysaccharide.

Non-limiting examples of cationic polysaccharide polymers include: cationic celluloses and hydroxy ethyl celluloses; cationic starches and hydroxyalkyl starches; cationic polymers based on arabinose monomers such as those which could be derived from arabinose vegetable gums; cationic polymers derived from xylose polymers found in materials such as wood, straw, cottonseed hulls, and corn cobs; cationic polymers derived from fucose polymers found as a component of cell walls in seaweed; cationic polymers derived from fructose polymers such as inulin found in certain plants; cationic polymers based on acid-containing sugars such as galacturonic acid and glucuronic acid; cationic polymers based on amine sugars such as galactosamine and glucosamine; cationic polymers based on 5 and 6 membered ring polyalcohols; cationic polymers based on galactose monomers which occur in plant gums and mucilages; cationic polymers based on mannose monomers such as those found in plants, yeasts, and red algae; cationic polymers based on the galactomannan copolymer known as guar gum obtained from the endosperm of the guar bean.

More preferably, said cationic polymer is a cationic polysaccharide, still more preferably said cationic polysaccharide is chitosan or a derivative thereof, even more preferably it is chitosan.

Advantageously, the chitosan or a derivative thereof is characterized by having a low molecular weight, such as of less than 90 kDa, less than 75 kDa, less than 50 kDa, less than 30 kDa, or less than 15 kDa.

Still advantageously, a derivative of chitosan is used wherein one or more hydroxyl groups and/or one or more amine groups have been modified (e.g., acetylated, alkylated or sulfonated chitosans, thiolated derivatives), with the aim of increasing the solubility of the chitosan or increasing the adhesive nature thereof.

Still advantageously, a derivative of chitosan is used with the aim of increasing the solubility of the chitosan or increasing the adhesive nature thereof.

In a preferred and advantageous embodiment of the invention, the particle obtainable by the method of the invention comprises (i) a protein-based shell, (ii) a hydrophilic or an amphiphilic biological compound; and said hydrophilic or amphiphilic biological compound is completely included in said protein-based shell.

In another preferred and advantageous embodiment of the invention, the particle obtainable by the method of the invention comprises (i) a protein-based shell, (ii) a hydrophilic or an amphiphilic biological compound; and said hydrophilic or amphiphilic biological compound is partially included in said protein-based shell and partially inside the particle.

In a preferred embodiment, the amphiphilic or hydrophilic biological compound at least partially included in the protein-based shell includes at least 75%, preferably at least 50%, more preferably at least 35%, or still more preferably at least 25%, of the total surface of compound.

In another preferred embodiment, the amphiphilic or hydrophilic biological compound at least partially included in the protein-based shell includes from 0.1% to 25% of the total surface of compound.

As described, the combination of the specific solvent and the protein chosen, allowed to have specific interactions, which distributed the biological hydrophilic or amphiphilic compound throughout the structure of the particle and not only in its core, thus enabling the particle of the invention to pass through the cell's membrane, deliver, and afterwards relieve the biological compounds, in several types of living cells, such as the bacterial cells, the mammalian cells and even the most reluctant ones, such as the plant cells.

Therefore, according to an aspect, the present invention provides a use of a particle, obtainable by the method of the invention, having the diameter in the range from 1 to 60 nm, for delivering and thus increasing bioavailability of a hydrophilic or an amphiphilic biological compound to a particularly reluctant organism, specifically plants cells.

According to another embodiment, the present invention provides a non-therapeutical cosmetic use of the particles obtainable by the method of the invention, having the diameter in the range from 70 to 700 nm for delivering and thus increasing bioavailability of a hydrophilic or an amphiphilic biological compound to animal cells, preferably mammalian cells, more preferably macrophage cells, even more preferably mouse cells.

Moreover, the invention relates to a method for treating a pathology which is addresses by the hydrophilic or amphiphilic biological compound of the particle of the invention, said method comprising a step of delivering a particle of the invention, comprising an efficacious amount of said hydrophilic or amphiphilic biological compound.

Because of its ability of internalizing, increasing the bioavailability and thus delivering said hydrophilic or an amphiphilic biological compound, the particle of the invention can be used in a method for the treatment of pathologies addressed by the hydrophilic or amphiphilic biological compound, in suitable amounts for treating the specific pathology, upon administration to living organisms, such as plants, bacteria, animals, preferably mammalians.

Thus, the invention further relates to a particle for use in delivering at least a hydrophilic or amphiphilic biological compound for the treatment of pathologies addressed by the hydrophilic or amphiphilic biological compound.

According to another aspect, the present invention provides a composition comprising a plurality of particles.

Advantageously, the composition is selected from the group consisting of a pharmaceutical composition, an agrochemical composition, an edible composition, and a cosmetic composition.

In fact, the composition of the invention is suitable for dietary, nutritional, dietetic or pharmaceutical use in mammals.

The composition of the invention can assume a wide variety of forms of preparation, according to the desired route of administration.

The composition of the invention may be in solid, liquid or semiliquid forms.

Preferably, the composition of the invention is in the form of a liquid.

More preferably, the composition of the invention in the form of a liquid, is stable from 3 to 10° C., still more preferably it is stable at 4° C.

Even more preferably, the composition of the invention in the form of a liquid is a water suspension.

In another advantageous and preferred embodiment, the composition of the invention is in solid form, preferably in the form of a powder, more preferably said powder is stable in a temperature range comprised from 4° C. to 25° C.

Advantageously, the composition comprises 1% to 80% (w/w), 1% to 50% (w/w), 1% to 25% (w/w), of the particles of the invention.

Preferably, the powder has a content of 0.5 mg/g to 500 mg/g of the particles of the invention.

In another embodiment, the powder has a content of 0.01 mg to 200 mg/g of the particles of the invention.

In a particularly advantageous embodiment, the composition of the invention has a polydispersity index in the range of about 0.05 to 0.7, preferably in the range from 0.2 to 0.6, preferably in the range from 0.2 to 0.3, as measured with Dynamic Light Scattering (DLS).

Advantageously, a composition as described herein has a zeta potential in the range of about 0 mV to 100 mV as measured with Dynamic Light Scattering (DLS).

Preferably, the composition of the invention has a zeta potential from 15-80 mV, more preferably 15-40 mV, still more preferably 15-30 mV.

In this embodiment the particle has a positive surface charge, being coated with a polysaccharide, such as chitosan, thus allowing specific interactions with the cells during the delivery.

In another embodiment, the composition of the invention has a zeta potential from −15 to −80 mV, more preferably from −15 to −40 mV, still more preferably from −20 to −30 mV.

In this embodiment the particle has a negative surface charge, being not coated with a polysaccharide, such as chitosan, thus allowing different interactions with the cells during the delivery.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXPERIMENTAL PART

Reference is now made to the following examples, which together with the above descriptions illustrate some embodiments of the invention in a non-limiting way.

In order to perform the following examples, the listed materials have been employed: Optipep® (whey hydrolyzed protein) was provided from Deimos Srl (Italy), Bovine Serum Albumin (BSA), Fluorescein isothiocyanate (FITC), chitosan low molecular weight and acetic acid were purchased from Sigma Aldrich (St. Louis, Mo., US), Green Fluorescent Protein (GFP) was produced and purified in house.

In order to perform the following examples, the listed methods for the particle size and surface charge analysis, have been employed: Z-average (mean diameter), zeta-potential (charge) and polydispersity index (PDI), have been employed: dynamic light scattering (DLS) principles using a Malvern Zetasizer (Nano-ZS; Malvern Instruments, Worcestershire, UK) at 25° C. Prior to the analysis, the samples were diluted 80 times to avoid multiple scattering effects.

### Example 1—Production of the Particle of the Invention Comprising a Protein-Based Shell, Different Hydrophilic or Amphiphilic Biological Compounds and an Optional Further Coating with Chitosan as Polysaccharide

1.1 Production of a Particle of the Invention Comprising a Protein-Based Shell of WPH and BSA (Bovine Serum Albumin) as Hydrophilic Biological Compound.

BSA was chosen as a model hydrophilic protein and a fluorescent labelled version (FTIC-BSA) was constructed, in order to track protein localization in plant cells.

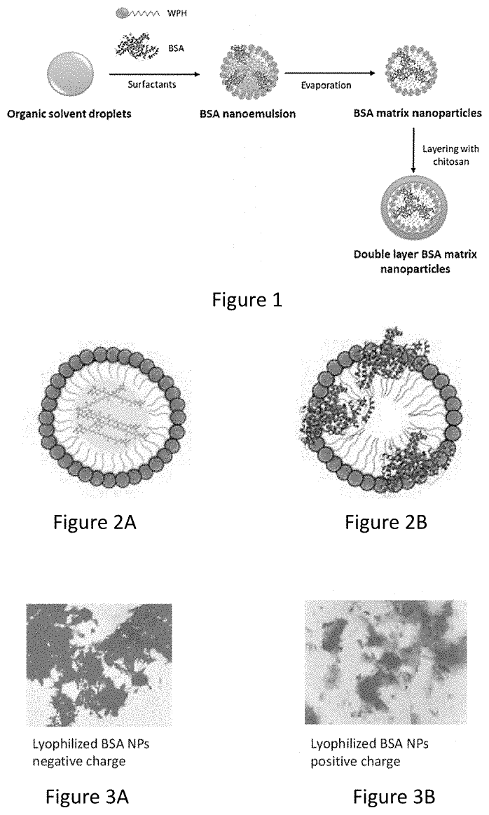

As shown in FIG. 1, in order to produce a particle having a shell of a protein, namely whey protein hydrolysate (WPH) and a hydrophilic biological compound, namely BSA (BSA-Ps), 630 mg of whey protein hydrolysate (WPH) and 270 of Bovine Serum Albumin (BSA) labelled with FITC or Alexa Fluor were left solubilize in 90 ml of deionized water under continuous stirring for at least 1 hour. After complete dissolution, the protein solution was mixed with ethyl acetate in the ratio 9:1 (protein solution/ethyl acetate), forming in this way a solution composed by two phases. A fine emulsion was produced sonicating the solution for 5 minutes at a potency of 10 W (Microson ultrasonic cell disruptor XL). The tube was kept in ice during sonication in order to avoid the overheating of the solution. At the end of the process, ethyl acetate was removed using nitrogen flow in the dark. BSA-WPH Ps were lyophilized via freeze drying and a dried particle comprising a shell of WPH protein and BSA biological hydrophilic compound, at least partially included in said protein-based shell, was obtained.

The specific type of encapsulation and encapsulating agents depend, among others, from the chemo-physical properties of the biological compound to be encapsulated, e.g. its affinity with polar aqueous chemicals. Specifically, this type of encapsulation is generally known and has been preferred for lipophilic biological compounds.

Differently from the case in which the active ingredient to be encapsulated was a lipophilic molecule, it is shown that the hydrophilic molecule, namely BSA, was included in the shell composed by WPH generating in this way a matrix-type particle, where the molecule of interest is distributed throughout the particle structure.

In fact, the shell at least partially surrounding a biological compound is in the form of a matrix structure or mold structure. The specific type of encapsulation depends, among other chemical interactions, from the chemo-physical properties of the active principle to be encapsulated, e.g. its value of hydrophobicity/lipophilicity and or hydrophilicity/lipophobicity. Specifically, this mold form of the protein-based shell is herein provided for a new type of encapsulation for hydrophilic or amphiphilic molecules.

FIG. 2A shows a representation of a known core-shell particle structure of the prior art, while FIG. 2B shows a representation of a matrix-type or mold-type particle structure of the invention.

1.2 Production of a Particle of the Invention Comprising a Protein-Based Shell of WPH, BSA (Bovine Serum Albumin) as Hydrophilic Biological Compound and a Further Coating of a Polysaccharide, Namely Chitosan.

In order to confer a positive charge to the surface of BSA-WPH Ps, 10 ml of the already formed BSA-WPH Ps solution was mixed with 2.8 ml of a chitosan 1% solution. The chitosan 1% solution was prepared using chitosan low molecular weight that is characterized by low viscosity, chitosan solubilization was carried out in warm deionized water with 1% acetic acid under stirring for some hours until complete solubilization, thus obtaining BSA-WPH particles coated with chitosan (chitosan-BSA-WPH Ps or BSA-WPH Ps⁺).

Thus, two types of BSA-WPH Ps with different surface charge were produced, in order to test whether or not this could affect the uptake of Ps by plant cells.

In the case of BSA-WPH Ps⁻ not coated with chitosan, the main population of particles had a diameter of 32.6±13 nm and a negative surface charge of −18±3.6 mV. The addition of a layer of chitosan (BSA-WPH Ps⁺) determined a slight increase in the average dimension of the NPs was 36.8±11.5 nm and a highly positive surface charge of 47.8±7.3.

Afterwards, both BSA-WPH Ps negatively and positively charged were freeze dried and the obtained powder characterized by a crystalline aspect is visible in FIG. 3A, representing negatively charged particles, thus BSA-WPH Ps⁻, and FIG. 3B, representing positively charged articles, thus BSA-WPH Ps⁺. The bright yellow color of the Figures was given by the presence of the labelling with FITC of BSA. The example confirmed that the particles were formed with BSA.

### Example 2—Cellular Testing of Delivery Using Positively Charged BSA-WPH Ps⁺ Vs Negatively Charged BSA-WPH Ps⁻ Particles of the Invention

The BSA-WPH Ps⁻ and BSA-WPH Ps⁺ particles, in the form of lyophilized powder as obtained according to Example 1, were used to determine the delivery of BSA-FITC into tobacco protoplast and intact tobacco cell suspension. BSA-WPH Ps⁻ had a dimension of 32.6 nm, measured by dynamic light scattering (DLS) principles using a Malvern Zetasizer (Nano-ZS; Malvern Instruments, Worcestershire, UK) at 25° C. and BSA-WPH Ps⁺ had a dimension of 37 nm.

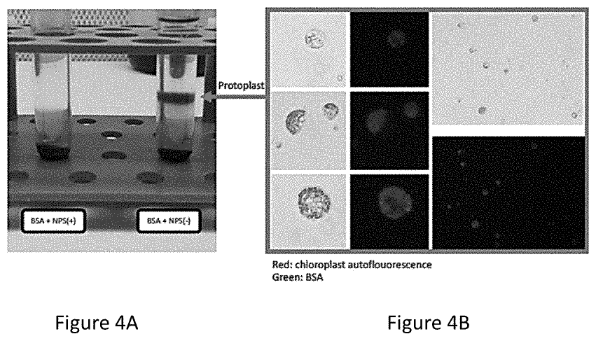

FIG. 4A shows a comparison which demonstrates that it was impossible to obtain protoplasts after the treatment with positively charged nanoparticles BSA-WPH Ps⁺, thus being coated with the further coating of chitosan, meanwhile it was possible to obtain protoplast when the cells were treated with BSA-WPH Ps⁻.

FIG. 4B shows that the negatively charged particle BSA-WPH Ps⁻, thus not being coated with the further coating of chitosan, was the only one internalized by tobacco protoplast, thus plant cells with the cell membrane but without cell wall. This type of cell has increased permeability due to the absence of the cell wall and an exposed plasma membrane, in that sense resembling an animal cell, allowing even negatively charged particles to enter.

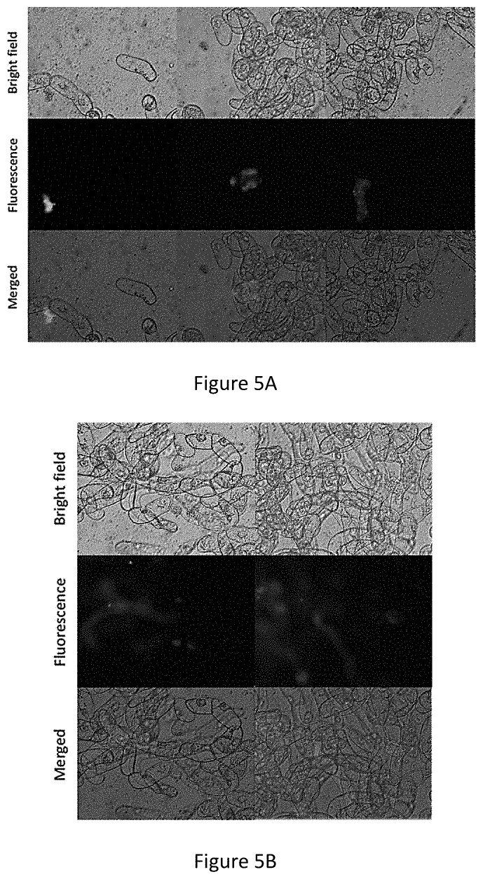

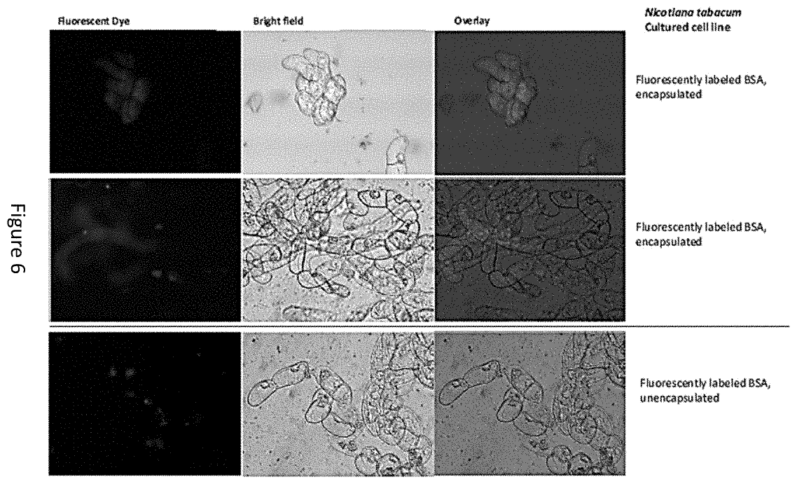

FIG. 5A is an optical microscope image, which shows the delivery of BSA-FITC using positively charged BSA-WPH Ps⁺ in tobacco cells, having both cell membrane and cell wall, it can be observed that the fluorescence observable in the picture is localized outside the cells, hence demonstrating that BSA-WPH Ps⁺ are not internalized, while FIG. 5B is an optical microscope image that shows the delivery of BSA-FITC using negatively charged BSA-WPH Ps⁻ and it is visible that BSA is internalized successfully within the tobacco plant cells.

Thus, it is possible to conclude that in case of entire tobacco plant cells, having both cell membrane and cell wall, the particles positively charged, thus having a further chitosan coating on the shell (BSA-WPH Ps⁺) cannot pass through not only the cell membrane but also the cell wall, while the negatively charged nanoparticles BSA-WPH Ps⁻ are able to internalize the biological hydrophilic compound, specifically BSA.

### Example 3—Delivery of the Particle of the Invention, Comprising a Protein-Based Shell of WPH, BSA (Bovine Serum Albumin) as Hydrophilic Biological Compound in a Plant Cell with Intact Plant Cell Walls

The delivery of the encapsulated biological compound, namely BSA, was examined using the plant species, Nicotiana tabacum.

Nicotiana tabacum cells, for example obtained from seedlings, can be propagated in suspension culture, where they retain intact cell walls. Encapsulated BSA in the particle of the invention (BSA-WPH Ps⁻), as obtained in Example 1, and non-encapsulated BSA were added to such cell suspensions. The BSA is linked to a fluorescent dye to enable visualization using fluorescence microscopy (as described under Example 1). The top two horizontal rows of FIG. 6 provide representative images of two experiments (biological replicates) with BSA-WPH Ps⁻. The bottom row shows the unencapsulated BSA control from one of these experiments.

The experiments confirm that the particle of the invention, namely BSA-WPH Ps⁻, is able to enter into the plant cells, penetrating the plant cell wall, hence causing internalization by these cells of the biological hydrophilic compound, specifically BSA. Furthermore, in the present example the inventors noted that particles of 5-50 nm and negative charge were successfully and optimally internalized in plant cells.

### Example 4—Size Distribution and Polydispersity (PDI) Analyses of the Particles of the Invention Comprising a Protein-Based Shell Made of Different Proteins and GFP (Green Fluorescent Protein) as Hydrophilic Biological Compound

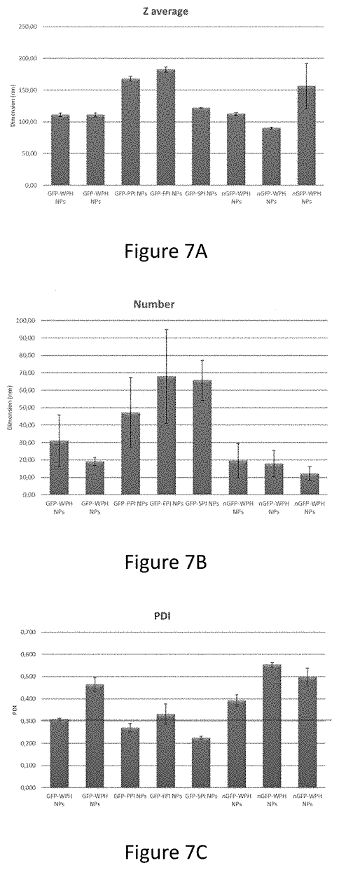

As shown in FIGS. 7A, 7B and 7C the graphs reported the z-average, the number (that is the real average diameter of the particles to be taken into account for this type of study) and the PDI index of the different particles produced according to Example 1, and having a shell comprising GFP (without or with a nuclear localization signal [NLS]) as hydrophilic biological compound and different proteins, such as WPH (OPTIPEP), fava protein isolate and soya protein isolate.

Said analysis has been working on a volume of 4 ml of reaction solution of the particles of the invention and sonicating for different times, specifically 1.5, 3 and 5 mins.

It can be seen from FIG. 7B that the average diameter of the particles of the invention always remains under the desired value of 60 nm, thus enabling them to be small enough to easily enter in the plant cells.

From FIG. 7C is possible to appreciate that the PDI index of the particles of the invention always remains in the range from 0.2 to 0.6, preferably in the range from 0.2 to 0.3, which is the desired range that assures stability in solution of the particles avoiding aggregation phenomena, thus reproducible delivery analysis when it comes to the delivery in plant cells.

### Example 5—Delivery Analyses of a Particle of the Invention Comprising a Protein-Based Shell of WPH and GFP (Green Fluorescent Protein) as Hydrophilic Biological Compound

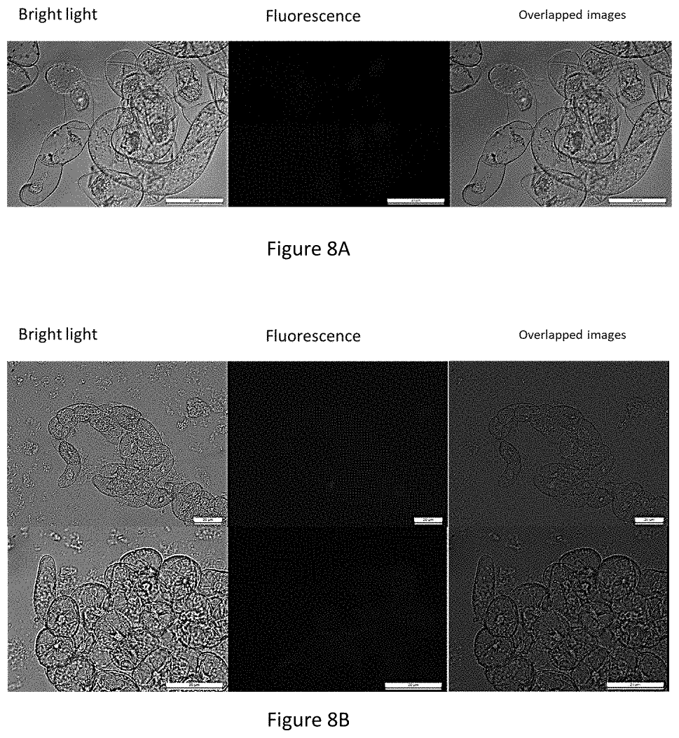

In order to examine the protein delivery through the particle of the invention, whey protein hydrolysate was used as protein constituting the shell, and the particles of the invention, GFP-WPH Ps were produced according to Example 1. The GFP-WPH Ps⁻ particles obtained had a particle size around 60 nm, thus being GFP-WPH NPs⁻ nanoparticles. Nicotiana tabacum cells in suspension were chosen and treated with the following preparations:
Non-treated cells (background fluorescent state, FIG. 8A);
Empty nanoparticles (NPs), with WPH in the shell, without GFP;
Non-encapsulated GFP NPs;
Encapsulated GFP-WPH NPs of the invention; and
Encapsulated GFP-WPH NPs of the invention, with nuclear localization.

For each sample, the fluorescence of the plant cells was examined though an optical fluorescence microscope after 24 and 48 hours and the following results were obtained and summarized in Table 1:

TABLE 1

| Type of nanoparticle | Presence of fluorescence |
|---|---|
| Control or Blank (Background | no |
| fluorescent state) | |
| Empty nanoparticles (NPs−) | no |
| Non-encapsulated GFP NPs− | no |
| Encapsulated GFP-WPH NPs− of the | yes |
| invention | |
| Encapsulated GFP-WPH NPs− of the | yes |
| invention with nuclear localization | |

The evidence summarized in Table 1 are further represented by FIGS. 8, 9, 10 and 11.

Specifically, FIG. 8A shows the background of the fluorescent signal given by non-treated cells, no fluorescence was observed. The fact that no fluorescence was observed, is an important data since sometimes, when cells are subjected to harsh condition (e.g. low amount of nutrient in the media of the suspension) they can still develop self-fluorescence, thus not being able to act as control in the experiments. In view of this, this experiment can be taken as blanc. FIG. 8B shows the results for the suspension of Nicotiana tabacum cells treated with empty nanoparticles (NPs), also here no fluorescence was observed.

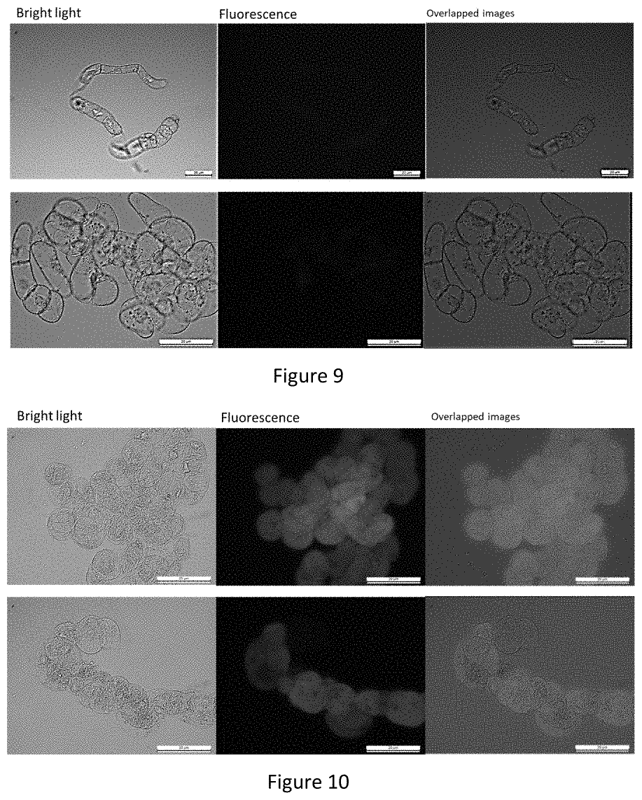

FIG. 9 shows the results for the suspension of Nicotiana tabacum cells treated with non-encapsulated GFP (naked GFP). No fluorescence was observed, confirming the fact that this molecule, namely non-encapsulated GFP cannot enter the cell but need to be delivered inside the cell through a cargo, otherwise it won't enter the plant cell, thus not being internalized.

FIG. 10 shows the results for the suspension of Nicotiana tabacum cells treated with encapsulated GFP-WPH NPs⁻. The latter were successfully up-taken by the cells, confirming that, when encapsulated via the method of the invention, the nanoparticles of the invention can pass though both the membrane cell and the wall cell of the plant cells.

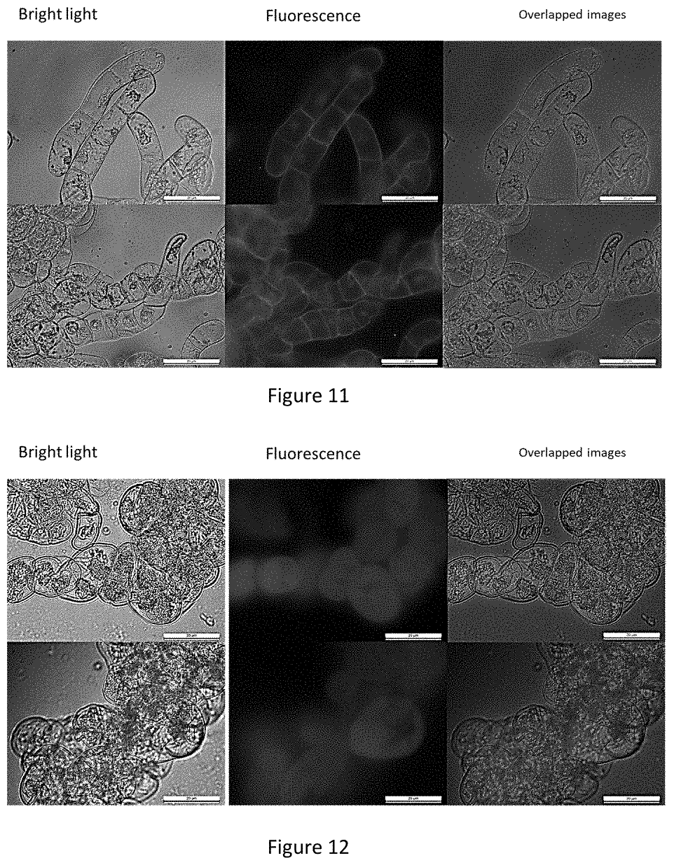

FIG. 11 shows the results for the suspension of Nicotiana tabacum cells treated with encapsulated GFP-WPH NPs⁻ of the invention with nuclear localization. The latter were successfully up-taken by the cells, confirming that, when encapsulated via the method of the invention, the particles of the invention comprising a shell including a protein, and a biological compound, can pass though the cell membrane, the wall cell and even reach the nucleus of the cells.

### Example 6—Delivery Analyses of a Particle of the Invention Comprising a Protein-Based Shell of Fava Isolate and Soya Protein and GFP (Green Fluorescent Protein) as Hydrophilic Biological Compound

In order to examine the protein delivery through the nano-encapsulated particle of the invention, GFP was encapsulated with Fava isolate protein and soya isolate protein, chosen as protein-based shell, and NPs with encapsulated Fava-GFP and soya-GFP were produced according to Example 1.

The Fava-GFP Ps⁻ obtained had a particle size of 67±26 nm, thus being Fava-GFP NPs nanoparticles. The soya-GFP Ps⁻ obtained had a particle size of 67±26 nm, thus being soya-GFP NPs nanoparticles.

Nicotiana tabacum cells from cell suspension and from solid calli were chosen and treated with the encapsulated Fava-GFP NPs⁻ nanoparticles. The results are presented, respectively, in FIG. 12 which show that the nanoparticles were successfully up-taken by the cells and they showed fluorescence inside the cells, thus confirming the possibility of using the same encapsulating method of the invention, with different protein-based shells.

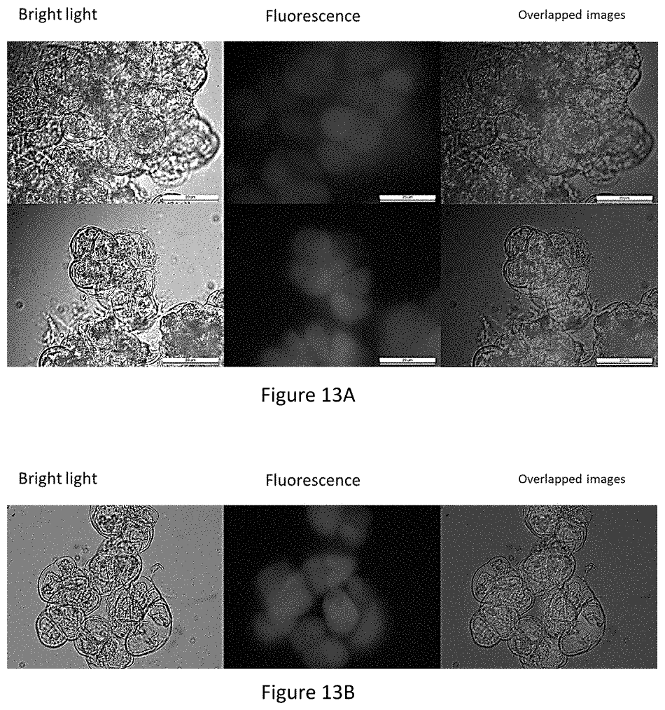

FIGS. 13A and 13B show, respectively soya-GFP NPs⁻ in cells suspension and calli, thus demonstrating the GFP internalization with different protein-based shell.

### Example 7—Delivery Analyses of a Particle of the Invention Comprising a Protein-Based Shell of Cellulase Enzyme+WPH and GFP (Green Fluorescent Protein) as Hydrophilic Biological Compound

In order to examine the GFP protein and enzyme delivery through the particle of the invention, 0.5 mg/ml of cellulase enzyme were used as protein in the shell along with WPH, and the particles of the invention, GFP-cellulase Ps were produced according to Example 1.

The cellulase-GFP Ps obtained had a particle size around 60-65 nm, thus being cellulase-GFP NPs⁻ nanoparticles.

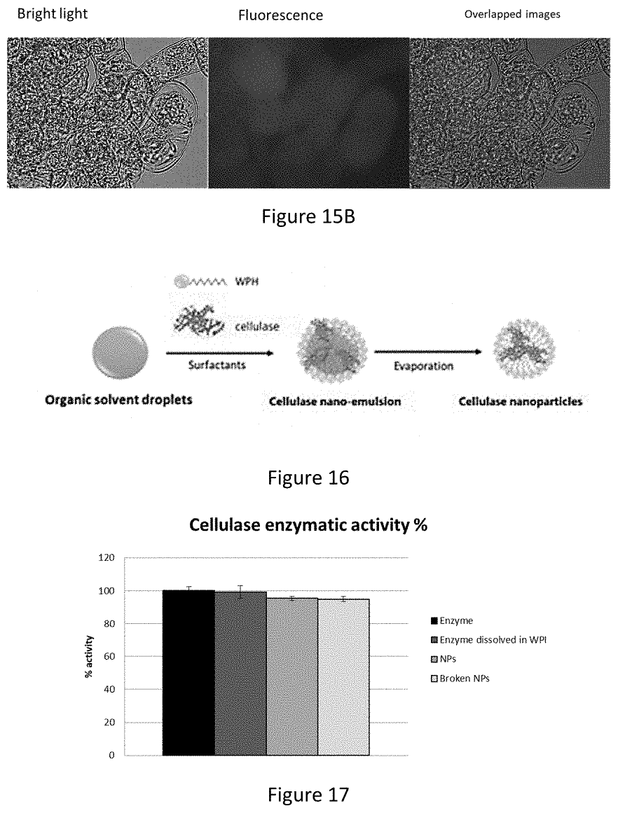

Nicotiana tabacum solid calli were chosen and treated, in suspension, with the empty nanoparticles and the encapsulated cellulase-GFP WPH NPs⁻. The results are presented in FIG. 15A and FIG. 15B, which show that the nanoparticles, encapsulating 0.5 mg/ml of cellulase on the shell and GFP as hydrophilic biological compound, were successfully up-taken by the cells and they showed fluorescence inside the cells, while the empty nanoparticles did not show any fluorescent, thus confirming the possibility of using the same encapsulating method of the invention also through enzymes with proteins as protein-based shell.

### Example 8—Delivery Analyses of a Particle of the Invention Comprising a Protein-Based Shell of WPH and DNA as Hydrophilic Biological Compound

In order to examine and demonstrate the DNA delivery through the particle of the invention, whey protein hydrolysate was used as protein in the shell, and the particles of the invention, DNA-WPH-Ps⁻ were produced according to Example 1.

The DNA-WPH Ps obtained had a particle size 55±37, thus being DNA-WPH NPs nanoparticles.

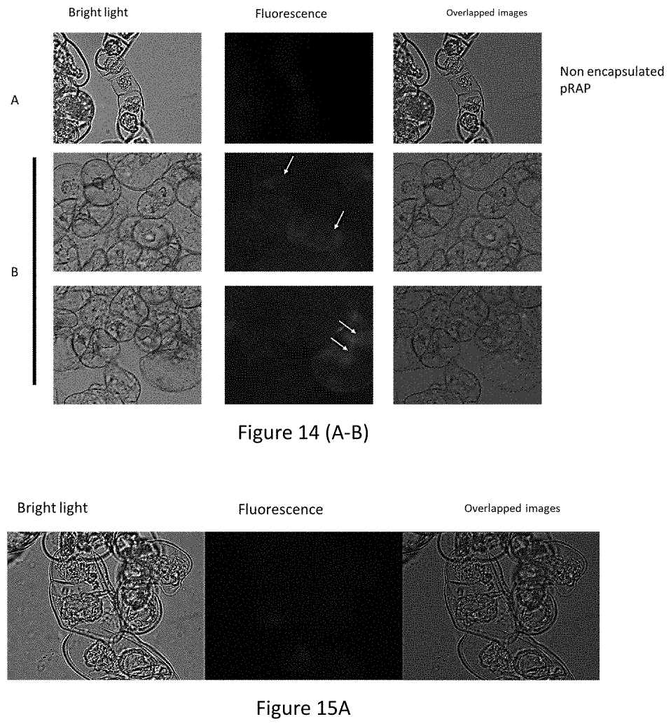

The delivered DNA consisted of a plasmid containing the GFP gene (with the Nuclear Localization Signal) under a strong promoter that can be used by the plant cell to trigger the expression. Nicotiana tabacum cells in suspension were chosen and treated, with the following preparations:
Non encapsulated pRAP (FIG. 14A)
Encapsulated pRAP NPs (DNA-WPH Ps⁻) (FIG. 14B) of the invention.

For each sample, the fluorescence of the cells was examined through an optical fluorescence microscope after 24 and 48 hours. FIG. 14A shows the absence of signal when the cells suspension was treated with non-encapsulated DNA, while FIG. 14B shows that pRAP-NPs (DNA-WPH Ps⁻), thus encapsulated DNA that expresses for GFP, were successfully up-taken by the cells and they showed fluorescence inside the cells, thus confirming the possibility of using the same encapsulating method of the invention, with different biological hydrophilic compounds. The arrows indicate the nuclear localization of the fluorescence inside the cells, as a consequence of the DNA expression.

### Example 9—Evaluation of the Retention of the Enzymatic Activity of Cellulase Enzyme after Encapsulation of a Particle of the Invention Comprising a Protein-Based Shell of WPH and Cellulase as Hydrophilic Biological Compound

In order to examine and demonstrate the retainment of the enzymatic activity after the encapsulation process of the particle of the invention, whey protein hydrolysate was used as protein in the shell, and the particles of the invention, cellulase-WPH-Ps were produced according to Example 1 and as schematically represented in the scheme in FIG. 16.

The cellulase-WPH Ps⁻ obtained had a particle size from 48 to 60 nm with a standard deviation of 6 nm, thus being cellulase-WPH NPs⁻ nanoparticles.

The following samples were prepared and subjected to the fenol solforic test performed directly on starch, to check the activity of the cellulase enzyme prior and after encapsulation in the particle of the invention:
Cellulase enzyme alone;
Cellulase enzyme dissolved in whey protein;
NPs of whey protein encapsulating cellulase, cellulase-WPH Ps− of the invention (NPs in FIG. 17); and
Enzyme activity retainment after encapsulation, where the NPs were broken (NPs broken).

It can be seen from FIG. 17, that, by comparing sample called “enzyme” and sample called “enzyme dissolved in whey protein”, the activity of cellulase was maintained also after the dissolution process, hence proving cellulase enzyme does not lose any activity after only dissolution with WPH protein.

Furthermore, FIG. 17 shows that, by comparing sample called “enzyme” and the sample called “NPs” (cellulase-WPH Ps⁻ of the invention), the activity of cellulase enzyme is only slightly reduced, thus confirming that the encapsulation method of the invention is able to produce a nanoparticle which retains the activity of the enzyme even after encapsulation.

Moreover, FIG. 17 shows that NPs of whey protein encapsulating cellulase, thus the particle cellulase-WPH Ps⁻ of the invention (“NPs” in FIG. 17) and the broken nanoparticles (“broken NPs” in FIG. 17), where the enzyme is supposed to be liberated in the supernatant and break down starch molecules, both showed to retain the same activity.

This evidence proves that the biological compound, namely the cellulase enzyme, is exposed on the surface and not physically blocked in the core of the particle, thus proving that the biological compound is included in the protein-shell, at least partially, so as the matrix-type structure of the particle of the invention is different from a core-shell particle.

### Example 10—Evaluation of the Retention of the Enzymatic Activity of Amylase Enzyme after Encapsulation of a Particle of the Invention Comprising a Protein-Based Shell of WPH and Amylase as Hydrophilic Biological Compound

In order to examine and demonstrate the retainment of the enzymatic activity after the encapsulation process of the particle of the invention, whey protein hydrolysate was used as protein in the shell, and the particles of the invention amylase-WPH-Ps⁻ were produced according to Example 1 and as schematically represented in the scheme in FIG. 16.

The amylase-WPH Ps obtained had a particle size from 40 to 56 nm, thus being amylase-WPH NPs⁻ nanoparticles.

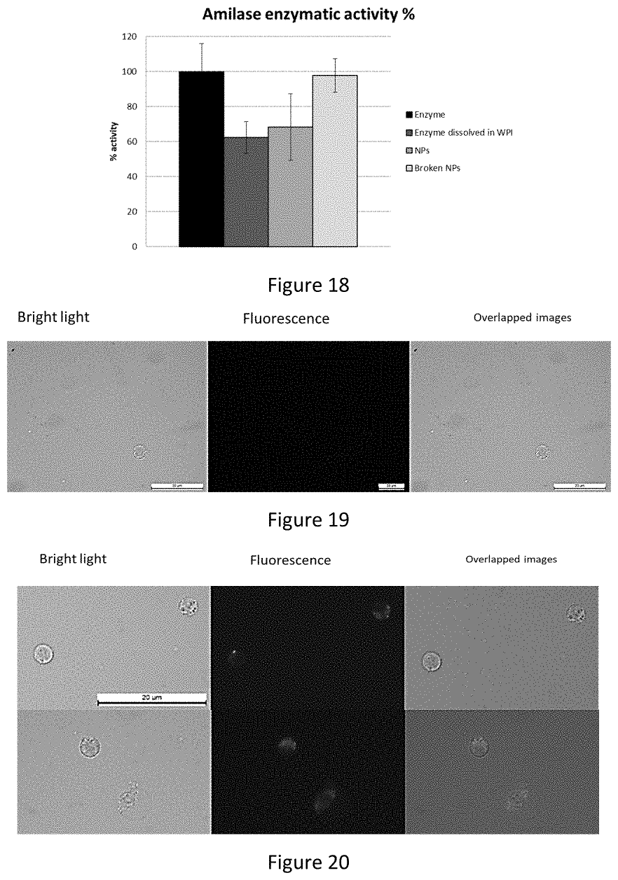

The following samples were prepared and subjected to the fenol solforic test performed directly on starch, to check the activity of the amylase enzyme prior and after encapsulation in the particle of the invention:
Amylase enzyme alone;
Amylase enzyme dissolved in whey protein;
NPs of whey protein encapsulating amylase, amylase-WPH Ps⁻ of the invention (NPs in FIG. 18); and
NPs broken.

It can be seen from FIG. 18, that, by comparing sample called “enzyme” and sample called “enzyme dissolved in whey protein”, the activity of amylase was highly reduced after the dissolution process, hence showing that the activity of amylase is not preserved when dissolved in WPH protein. In fact, the enzyme seems to self-interact with the protein, namely WPH, as showed by the decrease of the activity in the sample with the enzyme and the protein in solution.

Furthermore, FIG. 18 shows that, by comparing sample called “enzyme” and the sample called “NPs” (amylase-WPH Ps⁻ of the invention), the activity of amylase enzyme is reduced but still present, because the site of the enzyme is protected, thus confirming that the encapsulation method of the invention successfully retains the enzymatic activity after the encapsulation of the particle of the invention comprising a shell including them together with a protein, namely WPH.

NPs of whey protein encapsulating amylase, thus the particle amylase-WPH Ps of the invention (“NPs” in FIG. 18) showed a lower activity with respect to the broken NPs, where the enzyme is supposed to be liberated in the supernatant and break down starch molecules.

This evidence proves that the biological compound, namely the amylase enzyme, is partially exposed on the surface (partially included in the protein-shell) and partially inside the shell of the particle of the invention.

### Example 11 Delivery Analyses of a Particle of the Invention Comprising a Protein-Based Shell of WPH and GFP (Green Fluorescent Protein) as Hydrophilic Biological Compound

In order to examine the protein delivery in mammalian cells through the particle of the invention, whey protein hydrolysate was used as protein in the shell, and the particles of the invention, GFP-WPH Ps⁻ were produced according to Example 1. The GFP-WPH Ps⁻ particles obtained had a particle size around 60-70 nm, thus being GFP-WPH NPs⁻ nanoparticles. Cells of J774A.1 (murine macrophage) were treated with the GFP-WPH NPs⁻ suspension and observed after 30 minutes with a fluorescence optical microscope. Specifically, FIG. 19 shows the results for the suspension of cells not treated as a basal reference for fluorescence and FIG. 20 shows the ones treated with the nanoparticles (NPs) containing GFP. As can be observed by FIG. 20 GFP-WPH NPs⁻ were successfully up-taken by the cells, confirming that, when encapsulated via the method of the invention, the nanoparticles can be up-taken also by mammalian cells.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A method for encapsulating a hydrophilic or amphiphilic biological compound, comprising the steps of:
a. forming a two-phase solution by solubilizing the hydrophilic or amphiphilic biological compound and a protein in water to form a solution, and mixing the protein solution and a solvent, thereby obtaining a two-phase solution;
b. emulsifying the two-phase solution in order to obtain an emulsion; and
c. evaporating the solvent from the emulsion,
thereby obtaining a particle comprising: (i) a protein-based shell (ii) a hydrophilic or an amphiphilic biological compound,
wherein said hydrophilic or amphiphilic biological compound is distributed not only in the center of the particle, but said hydrophilic or amphiphilic biological compound is also at least partially included in said protein-based shell, and
wherein said solvent of step a. has a dielectric constant at 20-25° C. in the range from 1.5 to 15.

2. The method according to claim 1, further comprising a step e. of coating the particle with a polysaccharide.

3. The method according to claim 2, wherein said polysaccharide is a cationic polysaccharide, preferably selected from the group consisting of chitosan or a derivative thereof, wherein one or more hydroxyl groups and/or one or more amine groups have been modified (e.g., acetylated, alkylated or sulfonated chitosans, thiolated derivatives), more preferably is chitosan.

4. The method according to claim 1, wherein in step a. the solvent has a dielectric constant at 20-25° C. in the range from 1.8 to 6.02.

5. The method according to claim 1, wherein in step a. the solvent is selected from the group ethyl acetate, dichloromethane, pentane, chloroform, 1,4 dioxane, benzene, toluene, N-pentane, N-hexane, cyclohexane, preferably it is ethyl acetate.

6. The method according to claim 1, wherein in step a. the solvent is in a weight ratio between protein:solvent in the range from 10:0.5 to 8:1.5, preferably is in a weight ratio of 9:1 between protein:solvent.

7. The method according to claim 1, wherein in step a. said hydrophilic or amphiphilic biological compound is selected from the group consisting of an oligonucleotide, a nucleic acid, a protein, an enzyme, or any combination thereof.

8. The method according to claim 7, wherein said hydrophilic or amphiphilic biological compound is selected from the group consisting of BSA (bovine serum albumin), GFP (green fluorescent protein), RNA (ribonucleic acid), DNA (deoxyribonucleic acid), cellulase enzyme and amylase enzyme.

9. The method according to claim 1, wherein the protein of step a. is selected from the group consisting of whey protein, soya protein, pea protein, fava bean protein, potato protein or any combination thereof, preferably is selected from whey protein, fava bean protein, potato protein and soya protein.

10. The method according to claim 1, wherein the protein of step a. is in the form of a protein hydrolysate.

11. The method according to claim 1, wherein step b. of emulsifying is carried out by sonicating the solution for a duration of 5 minutes.

12. The method according claim 1, comprising a further step d. of drying the particle evaporated in step c. or the particle coated in step e.

13. The method according to claim 12, wherein the step d. of drying is selected from the group consisting of spray drying, granulating, agglomerating, freeze drying or any combination thereof, the particles.

14. The method according to claim 13, wherein said step d. of drying is carried out by freeze drying.

15. The method according to claim 1, wherein the particle evaporated after step c. or coated after step e. has an average diameter in the range from 1 to 60 nm, preferably from 5 to 60 nm, more preferably from 5 to 50 nm as measured with Dynamic Light Scattering (DLS).

16. The method according to claim 1, wherein the particle evaporated after step c. or coated after step e. has an average diameter in the range from 70 to 700 nm, preferably from 100 to 500, more preferably from 100 to 300 nm, still more preferably from 100 to 200 nm as measured with Dynamic Light Scattering (DLS).

17. A particle obtainable by the method according to claim 1, comprising:
(i) a protein-based shell; and
(ii) a hydrophilic or an amphiphilic biological compound,
wherein said hydrophilic or amphiphilic biological compound is distributed not only in the center of the particle, but said hydrophilic or amphiphilic biological compound is also at least partially included in said protein-based shell.

18. The particle according to claim 17, comprising a (iii) polysaccharide coating encapsulating the particle.

19. The particle according to claim 18, wherein said polysaccharide coating comprises a cationic polysaccharide, preferably said polysaccharide coating is a cationic polysaccharide, more preferably said polysaccharide is selected from the group consisting of chitosan or a derivative thereof, still more preferably it is chitosan.

20. The particle according to claim 17, having a diameter in the range from 1 to 60 nm, preferably from 5 nm to 60 nm, more preferably 5 nm to 50 nm as measured with Dynamic Light Scattering (DLS).

21. The particle according to claim 17, having a diameter in the range from 70 to 700 nm, preferably from 100 to 500, more preferably from 100 to 300 nm, still more preferably from 100 to 200 nm as measured with Dynamic Light Scattering (DLS).

22. The particle according to claim 17, wherein the protein-based shell comprises a protein selected from whey protein, soya protein, pea protein, fava bean protein, and potato protein or any combination thereof.

23. The particle according to claim 17, wherein the protein-based shell comprises a protein in the form of a protein hydrolysate.

24. The particle according to claim 22, wherein the protein-based shell comprises a protein selected from whey protein hydrolysate and fava isolate protein, still more preferably the protein-based shell comprises hydrolysate whey protein.

25. The particle according to claim 17, wherein the hydrophilic or amphiphilic biological compound is a hydrophilic compound selected from the group consisting of oligonucleotide, a nucleic acid, a protein, an enzyme, or any combination thereof.

26. The particle according to claim 25, wherein the hydrophilic or amphiphilic biological compound is selected from the group consisting of BSA (bovine serum albumin), GFP (green fluorescent protein), RNA (ribonucleic acid), DNA (deoxyribonucleic acid), cellulase enzyme and amylase enzyme.

27. A method for delivering of a hydrophilic or an amphiphilic biological compound in plant cells, comprising the step of using the particle according to claim 17.

28. A non-therapeutical cosmetic method for delivering at least a hydrophilic or amphiphilic biological compound inside animal cells, preferably mammalian cells, comprising the step of using the particle according to claim 17.

29. A method for delivering at least a hydrophilic or amphiphilic biological compound for the treatment of pathologies addressed by the hydrophilic or amphiphilic biological compound, comprising the step of using the particle according to claim 17.

30. A composition comprising a plurality of particles according to claim 17.

31. The composition according to claim 30, having a zeta potential from 15-80 mV, more preferably 15-40 mV, still more preferably 20-30 mV, as measured with Dynamic Light Scattering (DLS).

32. The composition according to claim 30, having a zeta potential from −15 to −80 mV, more preferably from −15 to −40 mV, still more preferably from −20 to −30 mV, as measured with Dynamic Light Scattering (DLS).

33. The composition according to claim 30, having a polydispersity index of 0.05 to 0.7, preferably in the range from 0.2 to 0.6, preferably in the range from 0.2 to 0.3 as measured with Dynamic Light Scattering (DLS).

34. The particle according to claim 23, wherein the protein-based shell comprises a protein selected from whey protein hydrolysate and fava isolate protein, still more preferably the protein-based shell comprises hydrolysate whey protein.
